(19) United States
(12) Patent Application Publication
(10) Pub. No.: US 2010/0281553 A1
(43) Pub. Date: Nov. 4, 2010

(54) PRODUCTION METHOD OF CYST EXPRESSED TRANSGENIC ANIMAL USING PKD2 GENE

(76) Inventors: Jong-Hoon Park, Seoul (KR); Han-Woong Lee, Gyeonggi-do (KR); Ji-Yeon Noh, Seoul (KR); Moon-Hee Yang, Seoul (KR); Yong-Hoon Sung, Gyeonggi-do (KR); Tae-Young Lee, Seoul (KR)

Correspondence Address: CANTOR COLBURN, LLP, 20 Church Street, 22nd Floor, Hartford, CT, 06103, US

(21) Appl. No.: 11/814,723

(22) PCT Filed: Nov. 3, 2006
(86) PCT No.: PCT/KR06/04551

§ 371 Date: Jul. 25, 2007

(30) Foreign Application Priority Data
Feb. 21, 2006 (KR) 10-2006-0016662

(51) Int. Cl. A01K 67/027; C12N 15/63

(52) U.S. Cl. 800/18; 800/24

(57) ABSTRACT

Disclosed herein is a method for producing a cyst-expressed transgenic animal using a PDK2 gene. The production method comprises preparing a PKD2 protein expression vector, inserting the expression vector into the nucleus of a fertilized egg to produce a PKD2 expression vector-containing fertilized egg, and transplanting the produced fertilized egg into the uterus of a surrogate mother. According to the invention disclosed herein, there is provided a method for producing transgenic animals, in which cysts are expressed only by the overexpression of the PKD2 gene. Also, transgenic mice are provided which can be effectively used in the investigation of cyst expression mechanisms and cyst control systems.

### TECHNICAL FIELD

The present invention relates to a method for producing a cyst-expressed transgenic animal using a PKD2 gene.

The inventive method for producing the transgenic animal comprises preparing a PKD2 protein expression vector, inserting the expression vector into the nucleus of a fertilized egg to produce a PKD2 expression vector-containing fertilized eggs, and transplanting the produced fertilized egg into the uterus of a surrogate mother to produce a cyst-expressed transgenic animal.

### BACKGROUND ART

The PKD2 gene is located on the long arm of chromosome 4 (4q21-23) and consists of 15 exons, and a protein expressed from the PKD2 gene was named polycystin 2.

Polycustin 2 is a molecule consisting of 986 amino acids and six transmembrane domains. It is known as ion channel that plays a role in calcium signaling, and interacts with polycystin 1 in cells.

To use such PKD2 to make mutations, including somatic inactivation, overexpression, and haploinsufficiency of PKD2, studies to find a cyst formation mechanism in transgenic animal studies are conducted.

In other words, it is known that the kidney and pancreas of mice having a disruption of exon 1 of the PKD2 gene have cysts observed therein and show cardiovascular defects and edemas.

Also, mice having a duplication of exon 1 of the PKD2 are transgenic animals having cysts formed in the kidney, liver and pancreas, and have been reported as animal models useful for the study of diseases caused by somatic inactivation (Biochem Biophys Res Commun 197: 1083-1093, 1993, Wu G et al., Cell 17: 93(2): 177-88, 1998).

Furthermore, it has been reported that mice from which exon 1 was removed using a LacZ promoter trap show characteristics, including randomization left-right patterning, right pulmonary isomerism), dextrocardia, and cyst formation in the kidney and pancreas, appear. The study of such models is useful for the study of the cyst formation mechanism (Proc Natl, Acad Sci USA 100: 5286-5291, 2003).

However, the cyst formation mechanism in transgenic animal models studied to date was not completely established. Thus, other transgenic models are required.

Korean Patent Registration No. 10-0494833 (entitled “liver cancer-expressed transgenic mice”) discloses liver cancer-expressed mice, which are produced by introducing an H-ras gene having a glycine-to-valine substitution at codon 12, and in which liver cancer is expressed as a result of the overexpression of the H-ras gene, so that they are useful for animal experiments in new drug development and can also be advantageously used in the study of mechanism of liver cancer development and the finding of novel gene involved in liver cancer development.

Korean Patent Registration No. 10-0434591 (entitled “human cancer-inhibitory gene, protein encoded thereby, expression vector containing the same, and cells transformed with the vector) discloses a human cancer-suppressing gene having a base sequence of SEQ ID NO: 1, useful for the prevention and treatment of human cancer, a protein encoded thereby, an expression vector containing the same, and microorganisms transformed with the vector.

Korean Patent Registration No. 10-0255582 (entitled “gene and genetic suppressor element associated with regulation of tumoral transformation in mammalian cells) discloses a genetic suppressor element of imparting the transformed phenotype of malignant mammalian cells in transformed cells, a method for identifying and obtaining said element, a method for identifying and isolating a gene corresponding to said genetic suppressor element, and the use of said genetic suppressor element.

However, studies on genetic diseases, including autosomal dominant polycystic kidney disease (ADPKD) causing complications such as renal failure, hepatic cyst, cerebral hemorrhage and valvular heart disease, are not yet conducted, and the mechanism of fluid filled cyst formation, which is the major characteristic of autosomal dominant polycystic kidney disease, is not yet known.

### DISCLOSURE

### Technical Problem

The present invention has been made to solve the above-described problems, and it is an object of the present invention to provide a method for producing a transgenic animal, in which cysts are expressed only by the overexpresion of a PKD2 gene.

Another object of the present invention is to provide a transgenic mouse which can be effectively used for the investigation of cyst expression mechanisms and cyst control systems.

### Technical Solution

The present invention relates to a method for producing a cyst-expressed transgenic animal using a PKD2 gene.

More particularly, the inventive method for producing the transgenic animal comprises the steps of: (1) preparing a PKD2 protein expression vector; (2) inserting the expression vector into the nucleus of a fertilized egg to produce a PKD2 expression vector-containing fertilized egg; and (3) transplanting the produced fertilized egg into the uterus of a surrogate mother to produce a cyst-expressing transgenic animal.

When the PKD2 gene, one of genes that cause autosomal dominant polycystic kidney disease, is mutated, a stop signal will not be passed to tubular cells, so that unlimited cell proliferation will progress, leading to the formation of a large number of cysts.

The autosomal dominant polycystic kidney disease is a relatively frequent genetic disease having a very high prevalence rate of 1 per 1,000 persons in the general population, forms innumerable cysts in both the kidneys, and causes complications such as renal failure, hepatic cyst, cerebral hemorrhage and valvular heart disease.

Also, cytogenesis is a phenomenon that is an important measure of the progression of ADPKD to renal failure, but a more fundamental therapeutic method of blocking the growth of cysts is required, and for this purpose, studies on cytogenesis, mutation analysis and the like are urgently required.

Thus, in the present invention, a PKD2 protein expression vector is injected into a fertilized egg, which is then the uterus of a surrogate mother, so that the PKD2 gene, one of ADPKD-causing genes, is overexpressed, thus producing a cyst-expressed transgenic animal.

As used herein, the term “mutant of the PKD2 protein” indicates a protein having at least one amino acid residue different from the wild-type amino acid sequence of PKD2, and refers to a protein having a sequence different from the natural amino acid sequence of PKD2, due to the deletion, addition, non-conservative or conservative substitution or combination thereof of at least one amino acid residue.

In some cases, the PKD2 protein can be modified by, for example, phosphorylation, sulfation, acrylation, glycosylation, methylation, and farnesylation.

Thus, the PKD2 protein shows an increase in the structural stability thereof against heat, pH and the like, or an increase in the activity thereof, due to the mutation or modification of the amino acid sequence thereof.

As used herein, the term “vector” indicates a means for introducing a nucleic acid sequence encoding a target protein into host cells.

Examples of the vector according to the present invention include a plasmid vector, cosmid vector and virus vector, and a suitable expression vector comprises, in addition to expression regulatory elements, such as a promoter, an operator, a start codon, a stop codon, a polyadenylation signal and an enhancer, a signaling sequence or reading sequence for membrane targeting or secretion, and can be prepared using various methods according to intended use.

Also, the expression vector comprises a selective marker for selecting host cells containing the vector, and when it is a replicable expression vector, it comprises a replication origin.

Thus, the vector used in the production of transgenic animals according to the present invention is preferably a vector, which enables the gene inserted therein to be irreversibly fused into the genome of host cells, such that the expression of the gene in the cells is stably continued for a long period of time.

Thus, when transgenic animals are produced using said expression vector, the produced transgenic animals can be used to conduct studies on a change in each tissue, and genes which are influenced by the overexpression of PKD2.

As used herein, the term “transgenic animals” means animals having a tumor generated by inducing the modification of characters such that the intracellular PKD2 protein level is increased compared to the normal cell level, and such transgenic animals have a high possibility to be used as tumor animal models.

As used herein “animal models” or “disease models” means models which are used to elucidate pathogenesis and pathophysiology using animals having a specific disease similar to human disease.

Thus, animals usable as animal models must enable the same effect as in the human beings to be predicted, must be easily produced, must be reproducible, and must show pathogenesis, which is the same as or similar to the pathogenesis of human disease.

Thus, suitable animal models are animals, which are vertebrate mammals, including human beings, and, at the same time, have the internal body structures (e.g., internal organs), immune system and body temperature similar to those of human beings, and suffer from disease such as hypertension, cancer, and immune deficiency.

Specifically, said animal is one selected from the group consisting of horses, sheep, pigs, goats, camels, antelopes, dogs, rabbits, mice, rats, guinea pigs, and hamsters.

Particularly, mice are most frequently used for the study of human diseases, because they are small prolific diseases, are genetically uniform, and can produce showing symptoms similar to diseases occurring in human beings.

A factor that determines the efficiency of animal model production by genetic manipulation is the screening of genes to be manipulated.

However, in the prior art, a method of overexpressing a single gene was also conducted in tumor animal models, but it was not easy to form cysts by the overexpression of the single gene.

The present inventors have found a method capable of forming cysts only by the overexpression of the PKD2 gene. In this method, when the PKD2 gene is overexpressed in cells, cysts are effectively generated in the kidneys of animals only by the overexpression of the PKD2 single gene without separation manipulation.

The overexpression of the PKD2 gene is achieved by introducing into cells a nucleic acid having a nucleotide sequence encoding the PKD2 protein. In this respect, the nucleotide sequence encoding the PKD2 protein is introduced in an amount sufficient for suppressing cell death, which is determined in consideration of the kind of animals or cells, the bodyweight of animals, and the mode and route f administration. The nucleotide sequence may be introduced in single or multiple doses.

The nucleotide sequence encoding the PKD2 protein is a nucleotide sequence encoding a wild-type or mutant-type PKD2 protein, can be mutated by the substitution, deletion, addition or combination thereof of at least one base, and can be isolated in nature or prepared using a chemical synthetic method.

Also, a nucleic acid having said nucleotide sequence can be single- or double-stranded, and may also be a DNA molecule (genome, or cDNA) or a RNA molecule.

Moreover, a nucleic acid sequence encoding the PKD2 protein is a nucleotide sequence encoding an amino acid sequence of SEQ ID NO: 2 (protein translated from cDNA of SEQ ID NO: 1).

The nucleotide sequence of SEQ ID NO: 1 can be exemplified by a nucleotide sequence encoding the amino acid sequence of SEQ ID NO: 2.

The human cDNA base sequence (2.9 kb) is shown in SEQ ID NO: 1, and the human PKD2 protein sequence (968 aa) is also shown in SEQ ID NO: 2.

Thus, the present inventors have conducted repeated studies on the basis of said invention and, as a result, could found that, when a method comprising preparing a PKD2 protein expression vector, inserting the expression vector into the nucleus of a fertilized egg to produce an expression-containing vector fertilized egg and transplanting the produced fertilized egg into the uterus of a surrogate mother to produce a transgenic animal is used, cysts are most effectively formed through the overexpression of the PKD2 gene in the transgenic animal.

Also, the present inventors could found that the transgenic animal produced by said production method can be effectively used in studies on gene functions and cyst formation mechanisms, and the research and development of cyst control systems.

Hereinafter, the inventive method for producing cyst-expressing transgenic animals using the PKD2 gene will be described in further detail.

1. Production Method of Transgenic Animals

(1) Step 1: Preparation of PKD2 Protein Expression Vector

A PKD2 protein expression vector is prepared.

Herein, it is preferable to prepare the expression vector by digesting a plasmid with restriction enzymes Xbal and Xhol (Promega) and introducing the PKD2 gene into the digested site.

Also, as the vector, it is preferable to use a pCAGGS vector which expresses a high level of a target protein from a beta-actin promoter. This vector is characterized in that it is stably maintained after insertion into host cells.

Thus, this vector was named “pCAGGS-PKD2 vector”.

The promoter of the expression vector pCAGGS-PKD2 consists of a fusion of a potent CMV promoter with a chicken β-actin promoter, which overexpresses the PKD2 gene in almost all tissues, but not in a specific tissue.

(2) Step 2: Production of Fertilized Egg

The expression vector is inserted into the nucleus of a fertilized egg to produce a fertilized egg.

Herein, the production of the fertilized egg is preferably conducted by removing cumulus cells from a fertilized egg resulting from hyperovulation induced by the administration of gonadotropin, and then injecting the expression vector directly into the nucleus of the fertilized egg using a microinjection needle.

(3) Step 3: Production of Transgenic Animal

The produced fertilized egg is transplanted into a surrogate mother to produce a transgenic animal.

Herein, the transgenic animal is preferably produced by transplanting the fertilized egg into the oviduct or implanting the fertilized egg into the uterus.

Also, the surrogate mother is one selected from the group consisting of horses, sweep, pigs, goats, camels, antelopes, dogs, rabbits, mice, rats, guinea pigs, and hamsters.

### ADVANTAGEOUS EFFECTS

According to the present invention, there is provided a method for producing a transgenic animal in which cysts are expressed only by the overexpression of the PKD2 gene.

Also, there is provided a transgenic mouse which can be effectively used in the investigation of cyst expression mechanisms and cyst control systems.

### DESCRIPTION OF DRAWINGS

FIG. 1 shows steps of forming cysts in a transgenic animal using the PKD2 gene according to the present invention.

FIG. 2 shows a recombinant expression vector (pCAGGS-PKD2) constructed by cloning the cDNA of PKD2 into a pCAGGS vector system.

FIG. 3 shows a photograph indicating the results of restriction enzyme cloning and a RT-PCR photograph indicating the expression of pCAGGS-PKD2.

FIG. 4 is a RT-PCR photograph of the genomic DNA of a transgenic mouse produced according to the present invention.

FIG. 5 is a photograph showing the results of Western blot analysis of a PKD2 protein expressed in each tissue of transgenic mouse lines A and B produced according to the present invention.

FIG. 6 is a photograph showing the results of Western blot analysis of a PKD2 protein expressed in the spleen and liver of transgenic mouse lines A-F produced according to the present invention.

FIG. 7 is an H&E photograph (scale bar: 50 μm) showing the generation of cysts in the kidney tissue of transgenic mouse line C produced according to the present invention.

*: cyst; and

[symbol] : glomerular cyst

FIG. 8 is an H&E photograph (scale bar: 50 μm) showing the generation of cysts in the kidney tissue of transgenic mouse line D produced according to the present invention.

*: cyst;

[symbol] : glomerular cyst; and

[symbol] : multinucleated cell.

FIG. 9 is an H&E photograph (scale bar: 50 μm) showing the generation of cysts in the kidney tissue of transgenic mouse line F produced according to the present invention.

↓ and [symbol] in FIG. 9A: cell showing hyperplasia;

↓ and [symbol] in FIGS. 9B and 9D: glomerular cyst;

C: high-magnification photograph of FIG. 9A; and

*: cyst.

FIG. 10 is a photograph (scale bar: 50 ↓m) showing the results of Masson's Trichrome staining, which show increases in inflammatory cells and collagen in the liver tissue of transgenic mouse line C produced according to the present invention.

[symbol] : increase in collagen;

Black: nucleus;

Red: cytoplasm or muscle;

Blue: collagen;

A and B: liver tissue of wild-type mouse line E; and

C and D: liver tissue of transgenic mouse line C.

FIG. 11 is a graphic diagram showing the total number and mean size of cysts found in 6-month-old mice and 18-month-old mice in transgenic mouse lines C, D, E and F produced according to the present invention.

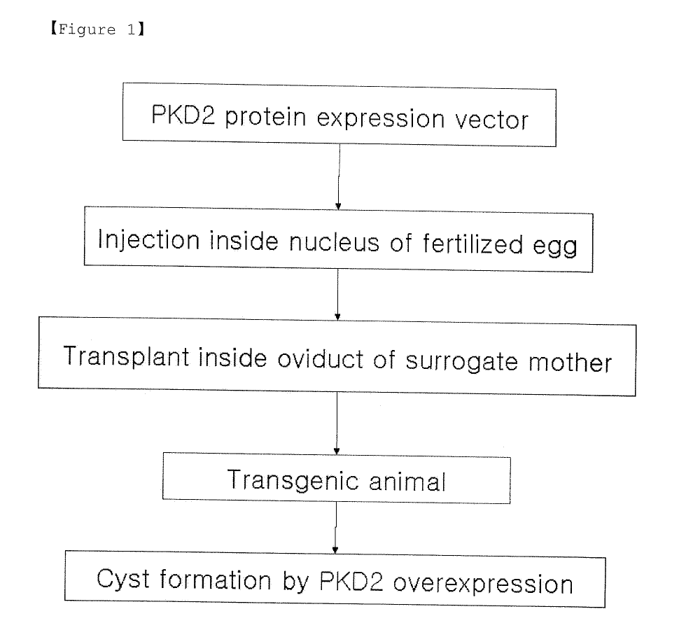

### BEST MODE

An inventive example for producing a cyst-expressed transgenic animal using a PKD2 gene is as follows.

### Example 1

### Production of Cyst-Expressed Transgenic Mice Using PKD2 Gene

1. Preparation of PKD2 Expression Vector

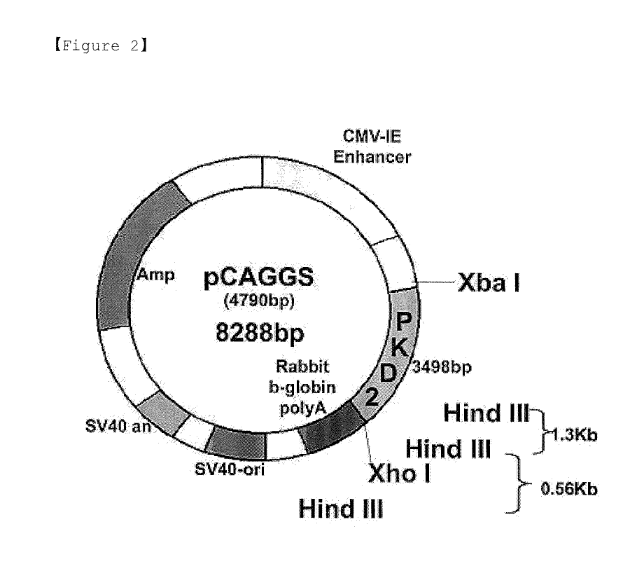

A pCAGGS plasmid (provided from Dr. niwa, RIKEN institute) was digested with two restriction enzymes Xbal and Xhol, and then inserted with human PKD2 cDNA (Genbank Accession #NM-000297) having a homology of 86% to mouse PKD2 in the protein level, thus preparing a pCAGGS-PKD2 plasmid recombinant vector (FIG. 2).

The prepared recombinant vector was purified with the midi-prep kit (QIAGEN) and used for the construction of a transgenic mouse.

2. Preparation of Fertilized Eggs

To obtain a large number of fertilized eggs, 5 IU of gonadotrophins such as PMSG (pregnant mare's serum gonadotrophin, ovulation induction, Sigma) and HCG (human menopausal gonadotrophin, follicle stimulation, Sigma) were injected intraperitoneally into inbred FVB/NJ female mice at 48-hr intervals, and then mated with EVE/NJ male mice. Then, the oviducts of the FVB/NJ female mice were collected.

To remove the surrounding cumulus cells, the oviducts were treated with hyaluronidase (mucopolysaccharidase, Roche), and 1-cell stage fertilized eggs were collected.

The fertilized eggs were washed with hyaluronidase in M2 medium (Sigma), transferred into M16 medium (Sigma), and stabilized in a 5% CO₂ incubator for 4 hours.

After completion of the stabilization, the PKD2 protein expression vector was injected into the fertilized eggs using a microinjector, and then the fertilized eggs were transferred onto M16 medium (Sigma) and incubated in a 5% CO₂ incubator for a given period of time, thus preparing PKD2 gene-containing fertilized eggs.

3. Production of Transgenic Mice

Among the incubated fertilized eggs, only fertilized eggs which differentiated from the 1-cell stage to the 2-cell stage were selected, and were transplanted into the oviducts of ICR (Institute of Cancer Research) surrogate mothers that were made pseudo-pregnancy by mating with ICR male mice that undergone vasectomy, thus producing transgenic mice of the present invention.

### MODE FOR INVENTION

Hereinafter, the present invention will be described in further detail with reference to examples and experiments. It is to be understood, however, that these examples are illustrative only and the scope of the present invention are not limited thereto.

### Example 2

### Production of Cyst-Expressed Transgenic Mice Using PKD2 Gene

1. Preparation of PKD2 Expression Vector

A pCAGGS plasmid (provided from Dr. niwa, RIKEN institute) was digested with two restriction enzymes Xbal and Xhol, and then inserted with human PKD2 cDNA (Genbank Accession #NM-000297) having a homology of 86% to mouse PKD2 in the protein level, thus preparing a pCAGGS-PKD2 plasmid recombinant vector (FIG. 2).

The prepared recombinant vector was purified with the midi-prep kit (QIAGEN), and a large amount of the purified PKD2 expression recombinant vector DNA was used for the construction of a transgenic mouse.

2. Preparation of Fertilized Eggs

To obtain a large number of fertilized eggs, 5 IU of gonadotrophins such as PMSG (pregnant mare's serum gonadotrophin, ovulation induction, Sigma) and HCG (human menopausal gonadotrophin, follicle stimulation, Sigma) were injected intraperitoneally into inbred C57BL/6 female mice at 48-hr intervals, and the female mice were then mated with C57BL/6. Then, the oviducts of the FVB/NJ female mice were collected.

To remove the surrounding cumulus cells, the oviducts were treated with hyaluronidase (mucopolysaccharidase, Roche), and 1-cell stage fertilized eggs were collected.

The fertilized eggs were washed with hyaluronidase in M2 medium (Sigma), transferred into M16 medium (Sigma), and stabilized in a 5% CO₂ incubator for 4 hours.

After completion of the stabilization, the PKD2 protein expression vector was injected into the fertilized eggs using a microinjector, and then the fertilized eggs were transferred onto M16 medium (Sigma) and incubated in a 5% CO₂ incubator for a given period of time, thus preparing PKD2 gene-containing fertilized eggs.

3. Production of Transgenic Mice

Among the incubated fertilized eggs, only fertilized eggs which differentiated from the 1-cell stage to the 2-cell stage were selected, and were transplanted into the oviducts of ICR (Institute of Cancer Research) surrogate mothers that were made pseudo-pregnancy by mating with ICR male mice that undergone vasectomy, thus producing transgenic mice of the present invention.

### Experiment 1

### Analysis of Overexpression of PKD2 Gene

Whether PKD2 cDNA was cloned during the preparation of the PKD2 expression vector in Example 1 was analyzed using two restriction enzymes Xbal and Xhol (Promega). To further confirm the cloning, the vector was additionally digested with HindIII (Promega), and as a result, it was found that the entire coding region (3890 kb) was transferred into the pCAGGS plasmid (FIG. 3).

Also, in order to examine whether the PKD2 expression vector used in the production of the transgenic mice in Example 1 was synthesized into a PKD2 protein in cells, the cells were transfected into mouse fibroblast cell line STO. RNA and a protein were extracted from the STO cell line, and whether the PKD gene was overexpressed in the cells was analyzed through RT-PCT and SDS-PAGE.

As a result, as shown in FIG. 3, the PKD2 expression vector used in the present invention was synthesized into the PKD2 protein in the cells, suggesting that the PKD2 gene was overexpressed. The base sequences of forward primers and reverse primers used in RT-PCR are shown in SEQ ID NOS: 3 and 4, respectively.

### Experiment 2

### Analysis of Germline Transmission of PKD2

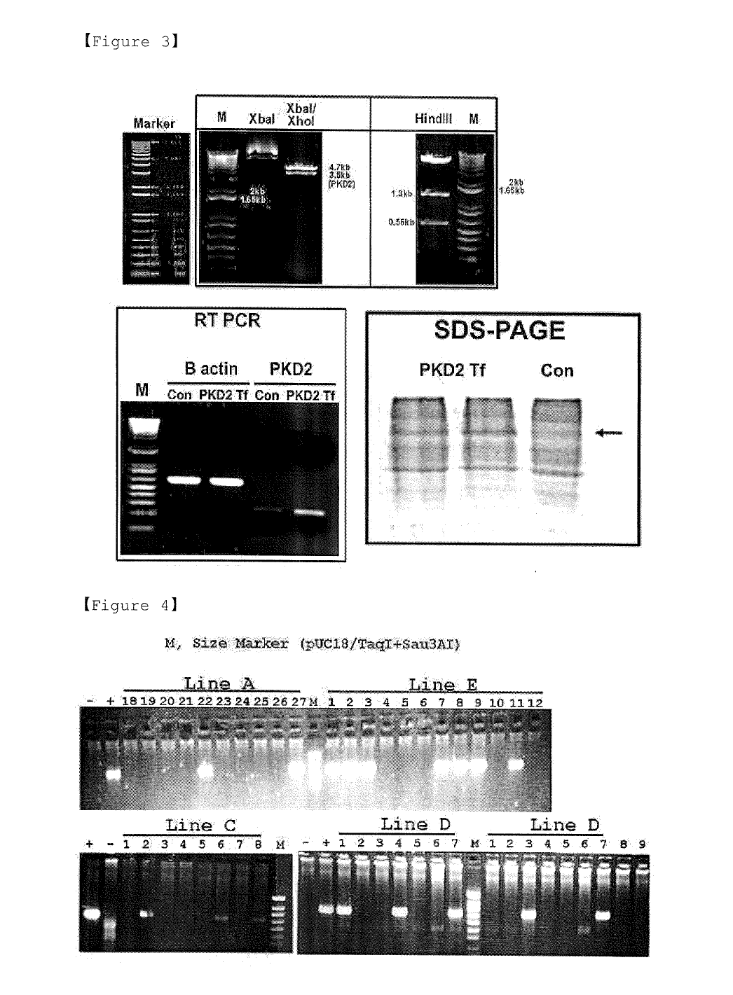

Genomic DNA was extracted from the tail or claw of newly born mice born 19 days after the production of the transgenic mice of Example 1, and was used to confirm the germline transmission of PKD2 through PCR (FIG. 4).

Herein, the PCR reaction for the confirming the germline transmission was conducted using forward and reverse primers, Tap polymerase conducted by overexpressing Taq from bacteria in this laboratory, and genomic DNA as a template. Also, the PCR reaction was performed in following conditions: denaturation of 5 min at 94° C., and then 30 cycles each consisting of 20 sec at 94° C., 25 sec at 55° C. and 45 sec at 72° C., followed by extension of 10 min at 72° C.

As a result, as shown in FIG. 4, the transgenic mice produced in the present invention showed the germline transmission. The base sequences of forward and reverse primers used in the PCR reaction are shown in SEQ ID NOS: 5 and 6, respectively.

### Experiment 3

### Measurement of Expression Level of PKD2 Gene

In the present invention, a total of six PKD2-overexpressed transgenic mice were produced, and were named mouse lines A-F for convenience.

On the basis of a characteristic in that, when a transgene is microinjected into transgenic mice, genomic DNA is expressed with recombination, the expression patterns of the transgene, which were different between the mouse lines depending on the recombined location, were observed. On the basis of the expression patterns, the characteristics of each of the mouse lines were analyzed.

In order to observe a pattern where the PKD2 gene is expressed into a protein, a protein was extracted from 5-week-old adult mice of mouse lines A-F and analyzed by Western blot.

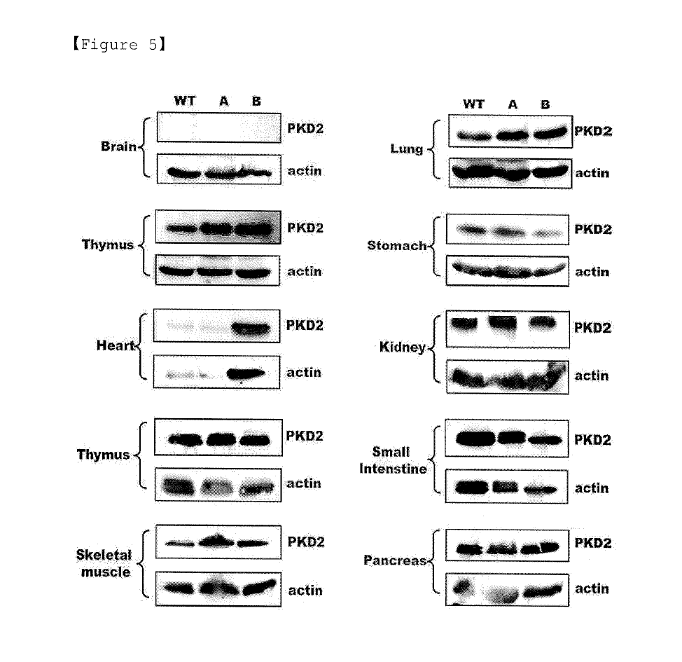

As a result, as shown in FIG. 5, similar expression patterns were observed in transgenic mouse lines A and B line, and the PKD2 protein was overexpressed in the lung and thymus tissues of the two mouse lines compared to the tissue of normal mice.

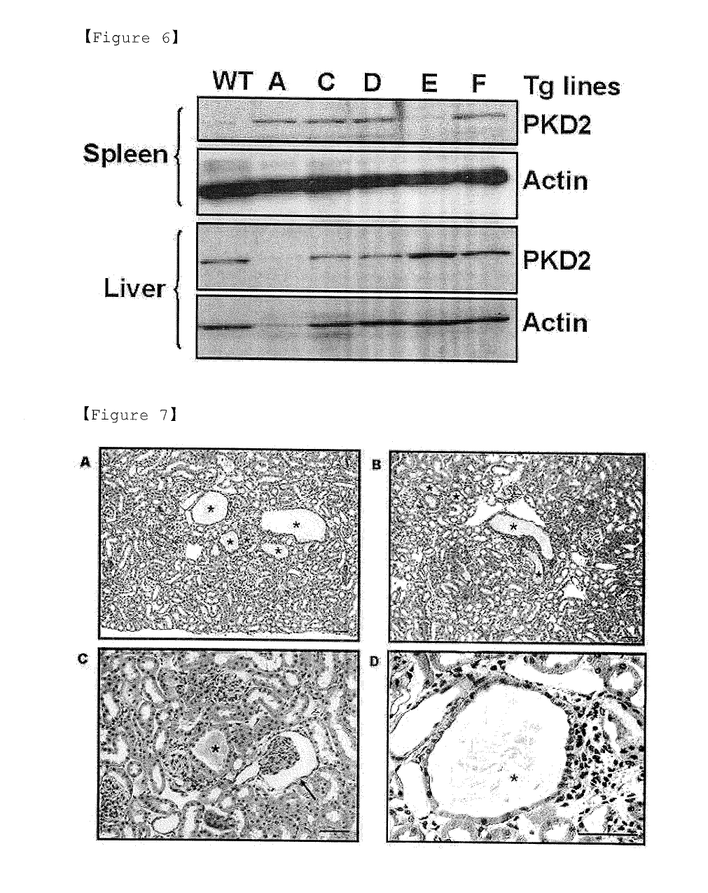

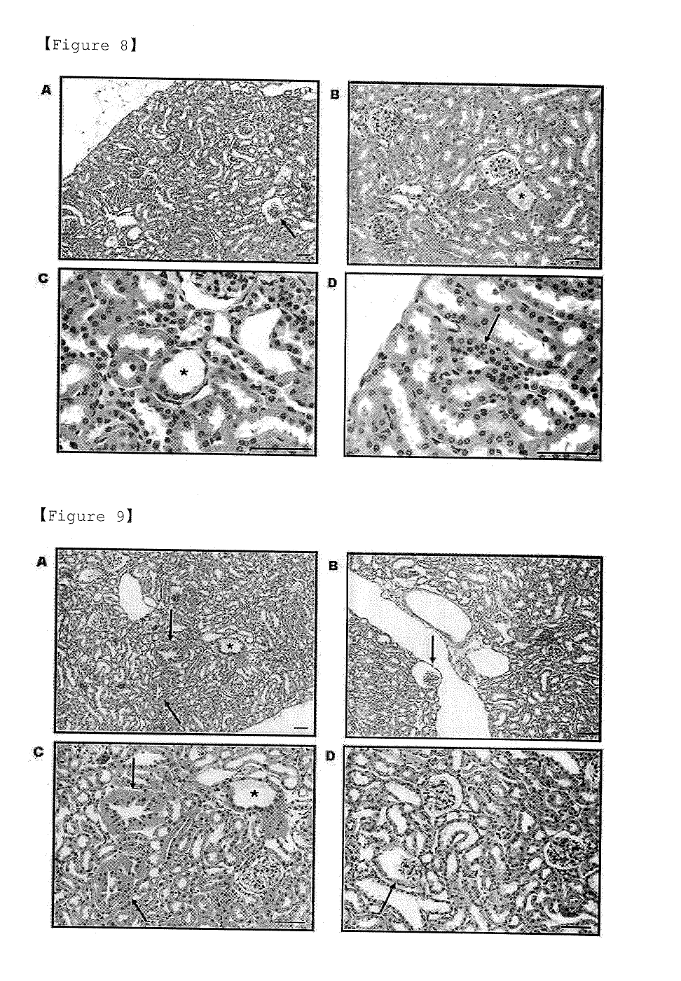

Also, as shown in FIG. 6, in the mouse lines other than the mouse line B, the overexpression of PKD2 appeared particularly in the spleen and liver tissues.

### Experiment 4

### Formation of Cysts in Kidney of PKD2-Overexpressed Transgenic Mice

The inventive transgenic mice produced in Example 1 were bred in a specific pathogen-free barrier system for 7 months (n=10). Then, the mice were sacrificed, and subjected to H&E (haematoxylin-eosin) staining.

The H&E staining results are shown in FIGS. 7, 8 and 9.

As shown in FIGS. 7, 8 and 9, various cysts, having various sizes, glomerular cysts and multinucleated cells were observed in the kidneys, and hyperplasia of a large number of the cells was shown around the cysts.

Also, a large number of lymphocytes were found in the interstitial tissue between tubules. This indicates that inflammatory reactions progressed.

In the regions where the glomerular cysts were formed, the local reduction of glomeruli, the enlargement of the Bowman's space, and the partial lack of glomerular tufts could be observed.

These changes in the shapes of the glomeruli and the Bowman's space would influence the structure and function of nephron. Also, in the epithelial cells of the formed cysts, various cells, including cuboidal cells, flat cells and apoptotic cells, were present. Such cells were consistent with intermediate stage cells suggested in “Tow hit model (Nishio S, J Clin Invest. Apr; 7(4):151-6, 2001)”. Thus, it is thought that the inventive transgenic mice will be of help in all studies ranging from the initial stage to progression stage of cyst formation.

### Experiment 5

### Observation of Increases in Inflammatory Cells and Collagen in Liver of Transgenic Mice

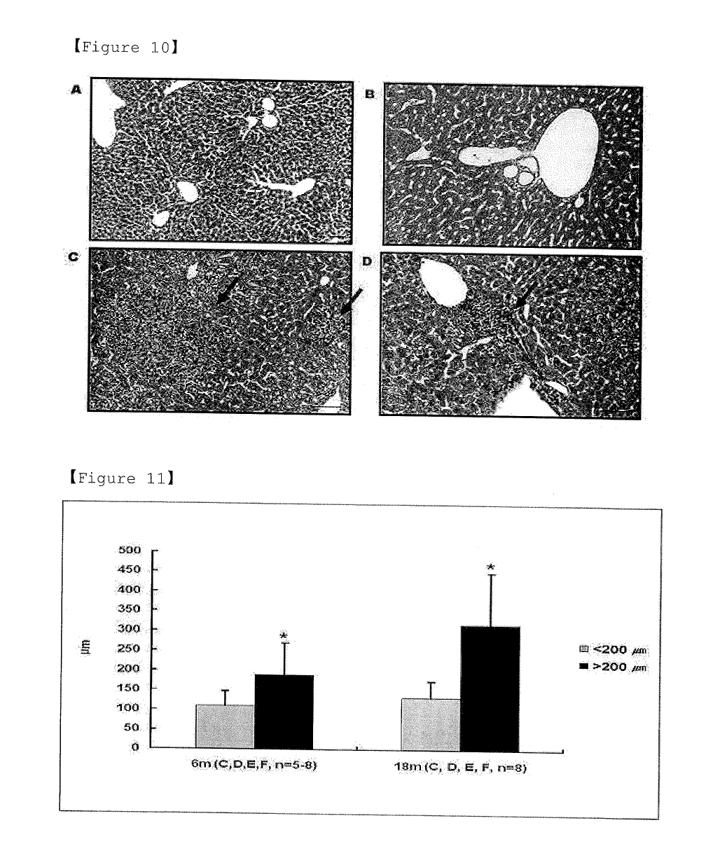

A large number of infected cells were observed around the biliary epithelium of liver of the transgenic mice produced in the present invention, and the observation results are shown in FIG. 10.

As shown in FIG. 10, the content of collagen in the liver was increased, suggesting the possibility of tissue fibrosis.

### Experiment 6

### Comparison of Total Cyst Number and Mean Cyst Size Between Four Transgenic Mouse Lines C, D, E and F

The cysts of four transgenic mouse lines produced in the present invention were observed. To examine the characteristics of each of the mouse lines, the mouse lines was were compared with each other with respect to total mean cyst size and the number and mean size of cysts having a size of more than 200 μm and cysts having a size of less than 200 μm.

The comparison results are shown in Table 1 below.

TABLE 1
Measurement of total number and mean size of cysts
found in transgenic mouse lines C, D, E and F produced in the
present invention

| | | Cyst size in a diameter (meam ± SD, μm) | | |
|---|---|---|---|---|
| | | Total | Cyst no. (mean size) | |
| PKD2 Tg | | mean size | M<200 μm | >200 μm |
| Line C (n = 14) | 6-18 | 130.04 ± 58.22 | 40 (128.37 ± 57.83) | 4 (250.72 ± 27.98) |
| Line D (n = 6) | 6-18 | 161.87 ± 97.50 | 10 (104 ± 31.30) | 5 (277.60 ± 77.16) |
| Ling E (n = 5) | 2-18 | 170.33 ± 84.7 | 12 (134.76 ± 37.61) | 4 (277.04 ± 102.18) |
| Line F (n = 3) | 7-18 | 257.59 ± 182.59 | 4 (151.35 ± 29.62) | 3 (399.24 ± 214.54) |

As shown in Table 1 above, the mean size of cysts found in the mouse line F was about two times as large as the other lines, and the number of cysts in the mouse line C was significantly larger than in the other mouse lines.

### Experiment 7

### Comparison of Total Cyst Number and Mean Cyst Size Between 6-Month-Old Mice and 18-Month-Old Mice Among Four Transgenic Mouse Lines C, D, E and F

The size and number of cysts were compared between 6-month-old mice and 18-month-old mice in four transgenic mice produced in the present invention to analyze the correlation between the age and cyst formation of the produced transgenic mice.

The analysis results are shown in Table 2 below and FIG. 11.

TABLE 2
Measurement of total number and mean size of cysts
found in transgenic mouse lines C, D, E and F produced in the
present invention

| | 6 month | |
|---|---|---|
| PKD2 Tg | (C, D, E and F, n = 5) | 18 month (C, D, E, F, n = 8) |
| <200 μm | 109.47 ± 38.09 (cyst n = 29) | 132.74 ± 4297 (cyst n = 28) |
| >200 μm | 190.27 ± 79.15 (cyst n = 4) | 318.47 ± 129.82 (cyst n = 11) |

As shown in Table 2 above and FIG. 11, the number and mean size of cysts having a size of more than 200 μm were significantly larger in the 18-month-old mice than in the 6-month-old mice, but in the case of cysts having a size of less than 200 μm, there was no great difference in cyst number and mean cyst size between the 18-month-old mice and the 6-month-old mice.

Thus, it was found that the formation of cysts having a size of more than 200 μm had a close connection with age.

### INDUSTRIAL APPLICABILITY

The present invention relates to the method for producing transgenic animals, in which cysts can be formed only by the overexpression of the PKD2 gene. According to the inventive method, when the PKD2 gene is overexpressed in cells, cysts can be effectively generated in the kidneys of animals with separate manipulation.

The inventive transgenic animals produced using the PKD2 gene can be used for the expression of fluid filled cyst, which is the major characteristic of autosomal dominant polycystic kidney disease (ADPKD) that causes complications such as renal failure, hepatic cyst, cerebral hemorrhage and valvular heart disease.

Also, the inventive transgenic animals can be effectively used in studies on gene functions and cyst formation mechanisms, and the research and development of cyst control systems.

1. A method for producing a cyst-expressed transgenic animal using a PKD2 gene, the method comprising the steps of:
(1) preparing a PKD2 protein expression vector;
(2) inserting the expression vector into the nucleus of a fertilized egg to produce a PKD2 expression vector-containing fertilized egg; and
(3) transplanting the produced fertilized egg into the uterus of a surrogate mother.

2. The method of claim 1, wherein the PKD2 protein expression vector is a pCAGGS-PKD2 plasmid vector prepared by inserting mouse PKD2 into a pCAGGS plasmid.

3. The method of claim 1, wherein the animal used in the step of the fertilized egg into the uterus of the surrogate mother is one selected from the group consisting of horses, sweep, pigs, goats, camels, antelopes, dogs, rabbits, mice, rats, guinea pigs, and hamsters.

4. A cyst-expressed transgenic mouse produced according to claim 1.

5. A cyst-expressed transgenic mouse produced according to claims 2.

6. A cyst-expressed transgenic mouse produced according to claims 3.


SEQUENCE LISTING (6 sequences)

SEQ ID NO: 1 - DNA, 2907 bp, Homo sapiens
atggtgaactccagtcgcgtgcagcctcagcagcccggggacgccaagcggccgcccgcgccccgcgcgccggacccgggccggctgatggctggctgcgcggccgtgggcgccagcctcgccgccccgggcggcctctgcgagcagcggggcctggagatcgagatgcagcgcatccggcaggcggccgcgcgggaccccccggccggagccgcggcctccccttctcctccgctctcgtcgtgctcccggcaggcgtggagccgcgataaccccggcttcgaggccgaggaggaggaggaggaggtggaaggggaagaaggcggaatggtggtggagatggacgtagagtggcgcccgggcagccggaggtcggccgcctcctcggccgtgagctccgtgggcgcgcggagccgggggcttgggggctaccacggcgcgggccacccgagcgggaggcggcgccggcgagaggaccagggcccgccgtgccccagcccagtcggcggcggggacccgctgcatcgccacctccccctggaagggcagccgccccgagtggcctgggcggagaggctggttcgcgggctgcgaggtctctggggaacaagactcatggaggaaagcagcactaaccgagagaaataccttaaaagtgttttacgggaactggtcacatacctcctttttctcatagtcttgtgcatcttgacctacggcatgatgagctccaatgtgtactactacacccggatgatgtcacagctcttcctagacacccccgtgtccaaaacggagaaaactaactttaaaactctgtcttccatggaagacttctggaagttcacagaaggctccttattggatgggctgtactggaagatgcagcccagcaaccagactgaagctgacaaccgaagtttcatcttctatgagaacctgctgttaggggttccacgaatacggcaactccgagtcagaaatggatcctgctctatcccccaggacttgagagatgaaattaaagagtgctatgatgtctactctgtcagtagtgaagatagggctccctttgggccccgaaatggaaccgcttggatctacacaagtgaaaaagacttgaatggtagtagccactggggaatcattgcaacttatagtggagctggctattatctggatttgtcaagaacaagagaggaaacagctgcacaagttgctagcctcaagaaaaatgtctggctggaccgaggaaccagggcaacttttattgacttctcagtgtacaacgccaacattaacctgttctgtgtggtcaggttattggttgaattcccagcaacaggtggtgtgattccatcttggcaatttcagcctttaaagctgatccgatatgtcacaacttttgatttcttcctggcagcctgtgagattatcttttgtttctttatcttttactatgtggtggaagagatattggaaattcgcattcacaaactacactatttcaggagtttctggaattgtctggatgttgtgatcgttgtgctgtcagtggtagctataggaattaacatatacagaacatcaaatgtggaggtgctactacagtttctggaagatcaaaatactttccccaactttgagcatctggcatattggcagatacagttcaacaatatagctgctgtcacagtattttttgtctggattaagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatcttcactcaattccgtatcattttgggcgatatcaactttgcagagattgaggaagctaatcgagttttgggaccaatttatttcactacatttgtgttctttatgttcttcattcttttgaatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatga

SEQ ID NO: 2 - protein, 968 aa, Homo sapiens
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTEADNRSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAACEIIFCFFIFYYVVEEILEIRIHKLHYFRSFWNCLDVVIVVLSVVAIGINIYRTSNVEVLLQFLEDQNTFPNFEHLAYWQIQFNNIAAVTVFFVWIKLFKFINFNRTMSQLSTTMSRCAKDLFGFAIMFFIIFLAYAQLAYLVFGTQVDDFSTFQECIFTQFRIILGDINFAEIEEANRVLGPIYFTTFVFFMFFILLNMFLAIINDTYSEVKSDLAQQKAEMELSDLIRKGYHKALVKLKLKKNTVDDISESLRQGGGKLNFDELRQDLKGKGHTDAEIEAIFTKYDQDGDQELTEHEHQQMRDDLEKEREDLDLDHSSLPRPMSSRSFPRSLDDSEEDDDEDSGHSSRRRGSISSGVSYEEFQVLVRRVDRMEHSIGSIVSKIDAVIVKLEIMERAKLKRREVLGRLLDGVAEDERLGRDSEIHREQMERLVREELERWESDDAASQISHGLGTPVGLNGQPRPRSSRPSSSQSTEGMEGAGGNGSSNVHV

SEQ ID NO: 3 - DNA, 24 bp, Artificial Sequence
  primer
tccctatggatttcactgtcacga

SEQ ID NO: 4 - DNA, 24 bp, Artificial Sequence
  primer
aggaggatgacgatgaagatagcg

SEQ ID NO: 5 - DNA, 20 bp, Artificial Sequence
  primer
tgctcagttggcataccttg

SEQ ID NO: 6 - DNA, 20 bp, Artificial Sequence
  primer
ctcctcagagtcatccaggc